(19) United States
(12) Patent Application Publication
(10) Pub. No.: US 2009/0112115 A1
(43) Pub. Date: Apr. 30, 2009

(54) Apparatus for detecting human's breathing

(75) Inventors: Jung-Tang Huang, Taipei (TW); Liang-Tse Lin, Taipei (TW)

Correspondence Address: Jung-Tang Huang, 5F., No.7, Lane 10, Sec. 2, Bade Rd., Da-an District,Taipei City 106 Taiwan (R.O.C.), Taipei, 106, TW

(73) Assignee: Jung-Tang Huang

(21) Appl. No.: 11/977,947

(22) Filed: Oct. 29, 2007

(51) Int. Cl. A61B 5/08

(52) U.S. Cl. 600/532

(57) ABSTRACT

An apparatus applied to detect the human breath gas, including a substrate which carries a circuit module, a CNT-based (carbon nanotube) gas sensing element, a wireless transmission/receiver module, and a power supplier, etc. and a clamp structure that could be clamped on the columella between the nostrils. The detectable gas also includes the bioaerosol in the gas. The CNT-based gas sensors react with the breath air and detect whether the specific gas and bioaerosol in the air or not while breathing in and out and the temperature of the air could be measured. This invention can measure the electric response of the CNT-based gas sensor, process the signal by the circuit module and transmit the processed signal to the wireless receiver by wireless transmission/receiver module. And the wireless signal receiver will differentiate the species, concentration and temperature of the gas and provide a warning signal while the specific gas or bioaerosol is detected. The apparatus is portable and has the functions of rapid response and high sensitivity.

### TECHNICAL FIELD

This invention relates to a gas and aerosol device for detecting human's breathing; it specifically relates to a sensor device that is hung around the nose end using carbon nanotubes for the detection of gas and aerosol; furthermore, it is relates to a device, which is in association with signal processing circuit and wireless transmitting/receiving module, for measuring, warning, transmitting and receiving physiological signal.

### BACKGROUND OF THE INVENTION

The gas exhaled from human body reflects the condition of organ and tissue in human body. For example, inflammation and oxidation stress can be monitored through the measurement of the concentration changes of the NO gas; the exhaled CO is a marker of cardiovascular diseases, diabetes, nephritis and bilirubin production; the exhaled low molecular weight hydrocarbon, for example, ethane and n-pentane, ethylene and isoprene; isoprene comes from the cholesterol synthesis process in human body and its concentration is related to the food; therefore, through the exhalation, the exhaled gas can be used as a special marker of the cholesterol concentration in the blood. [Reference: Karl T., Prazeller P., Mayr D., Jordan A., Rieder Fall J. R. and Lindinger, W., 2001, Human breath isoprene and its relation to blood cholesterol levels: new measurements and modeling, J. Appl. Physiol., 91, 762-70]

Acetone is a marker for diabetes; formaldehyde, ethanol, hydrogen sulfide and carbonyl sulfides shows the damage of the liver; however, for ammonia/amines—the later is a marker of renal diseases [Refer to literature by Smith A D, Cowan J O, Filsell S, McLachlan C, Monti-Sheehan G, Jackson P and Taylor D R, 2004, Diagnosing asthma: comparisons between exhaled nitric oxide measurements and conventional tests, Am. J. Resp. Crit. Care Med., 169, 473-8 and literature by Risby T H and Sehnert S S, 1999, Clinical application of breath biomarkers of oxidative stress status, Free Rad. Biol. Med., 27, 1182-92].

The odor of gas is due to infection and disorder, which provides a path for the application of chemical sensor in the biological field. The generation of NO₂ is related to the bronchial epithelial infection, which is caused by smoking. Besides, ammonia is a product of the decomposition of urea. [Studer S M, et al, 2001, Patterns and significance of exhaled-breath biomarkers in lung transplant recipients with acute allograft rejection, J. Heart Lung Transplant, 20, 1158-66].

Therefore, a sensor that can effectively monitor the gas exhaled by human being in the long term is very important. In addition, there are many contagious diseases or allergic diseases are from the external bioaerosol; if one is a carrier, the corresponding bioaerosol will be exhaled out of his body through mouth and nose and enters the external air; therefore, a device that can detect the gas exhaled from human body and aerosol is proposed in this invention, it more specifically relates to a sensor device that is hung around the nose end which uses carbon nanotubes for the detection of gas and aerosol; moreover, it is a device that can measure, warn and transmit and receive physiological signal in association with signal processing circuit and wireless transmitting/receiving module. Furthermore, the invented sensor through a way like a face mask, the gas and aerosol in and out of the mouth and nose can also be detected as long as the packaging method is changed accordingly.

### SUMMARY

This invention provides a device for detecting the gas inhaled and exhaled by human body and the detection includes the species, concentration, temperature and humidity of the gas inhaled and exhaled by human body; in the device, carbon nanotube is used as the sensor material; it is known that purified carbon nanotubes or surface-modified carbon nanotubes will react with specific gas to generate mass and electrical property change, for example, resistance, capacitance, and transistor characteristics; for foreign hazardous gas, the features of carbon nanotube sensor such as high sensitivity and high response speed can be used to inform the user to escape from the environment once hazardous substances are detected so as not to be injured by the hazardous gases, for example, CO and methane, etc.; moreover, it can detect species, rate of change of concentration, temperature and humidity of the gas exhaled by human body to be used as reference for monitoring physiological status or the purpose of diagnosis. If we perform further adsorption modification by using DNA or antibody or aptamer or carbohydrate on carbon nanotubes, we can detect the aerosol inhaled or exhaled by human body, for example, the detection of flu virus and tuberculosis bacteria, etc.

A device that can achieve the objective of the above mentioned invention for detecting the gas and aerosol exhaled by human body comprising of at least: a substrate, a carbon nanotube gas sensor device, a signal processing circuit, a wireless transmitting/receiving module or a body-network transmitting/receiving module, and a power supply.

The user can wear the device for detecting the gas and aerosol inhaled and exhaled by human body and the device can continuously detect the gas and aerosol inhaled and exhaled by the user; after the reaction of carbon nanotube sensor device with the gas or aerosol, a signal processing circuit will be used for the electrical measurement of carbon nanotube sensor device; then the electrical signal measured will be transmitted to a remote monitoring device or another warning device through wireless transmitting/receiving module or a body-network transmitting/receiving module for the monitoring or recording purpose as required by a user or a monitoring person; if the user has been detected with the inhalation or exhalation of hazardous gas or aerosol, high inhaled or exhaled temperature or the exhaled gas has very low content of water which is suspicious of dehydration, then the user or the remote monitoring personnel can get the warning immediately.

### BRIEF DESCRIPTION OF DRAWINGS

The invention may best be understood by reference to the following description taken in conjunction with the accompanying drawings that illustrate specific embodiments of the present invention.

FIG. 1 is a system architecture of the present invention for a gas and aerosol detection device to detect the gas and aerosol inhaled or exhaled by human body.

FIG. 2 is a detection device of the present invention that can be used to detect the gas and aerosol inhaled and exhaled by human body and is hung around the nose outer wall of human body.

FIG. 3 illustrates carbon nanotube sensor device of the present invention.

FIG. 4 is an embodiment of the device of present invention clamped on the columella between the nostrils of human nose.

FIG. 5 is the experimental result of the present invention: wherein cross-linking agent is used to modify antibody onto the surface of carbon nanotube and carbon nanotube transistor is used to detect biological particle; when the biological particle is adhered to the surface of the carbon nanotube, the electrical property change is measured, that is, I_{sd}−V_{gs} characteristic diagram is drawn.

FIG. 6 is the experimental result of the present invention: When PBS mixed solution with salmonella is dropped, it is found immediately that there is an obvious drop in the current, when it drops to about 1.4×10⁻⁶ A, it will restore to a stable status; later on, add other types of cells (Pseudomonas aeruginosa) into the buffer solution, the current won't change. Therefore, through such an electrical signal experiment, it is found that the reaction of the combination of salmonella with the corresponding antibody will cause an obvious drop in the electrical conductivity of the carbon nanotube.

FIG. 7 is the experimental result of the present invention: Wherein CNTFET is used as biomedical sensor (ssDNA) and gas sensor (acetone). (a) is the titration of “A” basic ssDNA, “ON” current will rise and the I_{sd}−V_{gs} curve will shift toward “positive” direction; (b) is the titration of “T” basic ssDNA, “ON” current will drop and I_{sd}−V_{gs} curve will shift toward “negative” direction; (c) is the titration of “C” basic ssDNA, “ON” current will drop and I_{sd}−V_{gs} curve will shift towards “positive” direction; (d) is the titration of “G” basic ssDNA, “ON” current will drop; (e) is the real time measurement of acetone CNTFET sensor surface-modified with DNA.

FIG. 8 shows that droplet that contains flu vaccine will approach CNTFETs chip easily due to the suction action (for example, the suction action performed by the human nose); when it gets in contact with flu antibody or flu aptamers on multiple single-wall carbon nanotubes (only single nanotube is illustrated in the figure), binding reaction will be generated within the droplet.

### DETAILED DESCRIPTION

Nano sensor device has been widely discussed and researched in recent years mainly because the nano sensor device has the advantages such as high sensitivity and low power consumption; it is especially useful for the application in the biomedical detection field, for example, for bacteria, virus or DNA with dimension smaller than sub-micrometer or even down to nano scale. The dimension of the object to be tested is much smaller than the sensor device dimension of MEMS device. Therefore, sensor device that is made using micron scale can not meet the demand in the detection accuracy and speed. In the present invention, carbon nanotube having semiconductor characteristics is used as nano sensor device (nanosensor) to perform the detection of substance that is harmful to the human body and the monitoring of human health status so as to achieve the purpose of high sensitivity, low energy consumption, capability of repeated measurement and low manufacturing cost; furthermore, this sensor unit can be used massively and at the same time in the environmental monitoring and human health status monitoring; moreover, if there are more people using it, it can be linked together as a wireless sensor network and a large and dead-corner-free protection net is thus formed and besides, it is mobile. This is especially true in the eruption of epidemic disease, for example, avian flu, foot-and-mouth disease, mad cow disease, SARS, etc.; during that period of time, it can be embedded into part of the fowls, pigs or cows and the patient that is suspicious of SARS disease; if it can be further added into wireless sensor network, the protection breadth and density is going to be greatly enhanced.

In recent years, many research organizations continuously get involved in related researches on electronic device based on carbon nanotube, the research results shows that carbon nanotube electronic device, under shorter channel length, for example, smaller than one micron, will have the characteristic of ballistic transportation; meanwhile, a single carbon nanotube channel can take current of ˜25 μA, and all these superior transistor characteristics could make it replace the present CMOS chip and become the electronic device of the next generation. In addition, carbon nanotube electronic device, when the channel is of longer length, can be used to detect the foreign molecules in the environment (gas molecules and biological molecules, etc.); it not only has high sensitive detection capability, but also has very small detector volume and low power consumption; moreover, after special carbon nanotube surface modification, it can be used as a sensor device that has high sensitivity and is dedicated for specific detection. From the above introductions, it can be seen that electronic device based on carbon nanotube will become potential transistor and sensor device in the future.

According to a study performed by Kong et al. in 2000 [see: Science 287, 622 (2000)], when semiconductor type carbon nanotube is exposed to gas molecules, for example, NO₂, NH₃ and O₂, its resistance value will be changed, and its response speed is about 10 times that of the conventional solid-state sensor; under room temperature, semiconductor type single wall carbon nanotube will have a sensitivity on gas of over 10³.

Due to the high sensitivity of carbon nanotube, in order to prevent the detecting result being affected by the environment, for example, temperature and humidity, there are several solutions provided in many researches, for example, Ashish Modi et al. in 2003 [Letters to Nature, VOL 424, 10 Jul. 2003] tried to use carbon nanotube as gas molecule sensor; different gas will have different breakdown voltage and current to distinguish gas species and concentration, and most importantly, it is not affected by the environment. In the device, there is one aluminum cathode and one anode made up of vertical and multi-wall carbon nanotubes on the SiO₂ substrate through chemical vapor deposition (CVD) method (with diameter of 25˜30 nm, length of 30 nm and spacing of 50 nm) for the detection of different gases in the air; the research result shows that the sensor has very good gas selectivity and sensitivity.

In addition, Snow et al. in 2005 [Science 307, 1942 (2005)] had proposed a gas detecting mechanism through the use of the measurement of the capacitance value of single wall carbon nanotubes when polarizing the gas nearby, which not only has higher sensitivity but also has larger concentration measurement range. Penza et al. in 2004 [Sensors and Actuators B 100, 47 (2004).] had proposed the deposition of one layer of carbon nanotube on the Surface Acoustic Waves (SAWs) sensor to be used as the detecting device of volatile organic mixed gas, for example, ethanol, ethyl acetate, toluene, etc with result showing high sensitivity. Ong et al. in 2002 [IEEE Sens. J. 2, 82 (2002)] had proposed the use of mixing thin film of SiO₂ and multi-wall carbon nanotube as gas sensor, which used the measurement of thin film capacitance and dielectric constant to judge the gas absorbed. Wong et al. in 2003 [Proc. IEEE Int. Symp. Circuits Sys. 4, IV844 (2003)] used AC electrophoresis force to manipulate multi-wall carbon nanotube, which was crossed and connected to Au micro electrode, to be used as temperature sensor; through the continuous measurement of voltage (V) and current (I), the result showed that the energy consumption was only in the range of several μWs.

Chopra et al. in 2002 [Appl. Phys. Lett. 80, 4632 (2002)] had deposited single-wall and multi-wall carbon nanotube on the microwave resonant sensor for the detection of ammonia. Someya et al. in 2003 [Nano Lett. 3, 877 (2003)] had used single-wall carbon nanotube field effect transistor (FETs) for the detection of ethanol vapor, when the surface of carbon nanotube absorbs ethanol vapor and gets saturated, the current value measured is going to drop rapidly to a fixed value. Staii et al. in 2005 [Nano Lett. 5, 1774 (2005)] had used single-wall carbon nanotube filed effect transistor as a sensor device and found that it could be used to detect different gases with very rapid response speed and response time; when it was exposed at different gas, it would generate different detecting current; meanwhile, this sensor device had the capability to restore to its detecting capability, and after of a detecting cycle of more than 50 repeated times, it still had very good detecting capability.

Currently, there are many researches that use carbon nanotube as sensor device, and the gases that have currently been verified to be able to be detected by carbon nanotube include: NH₃, CO₂, O₂, NO₂, CH₄, H₂, N₂, Ar, CO, NO, He, SF₆, methanol, ethanol, Organophosphorus pesticides, etc.

### Carbon Nanotube Gas Detecting Principle

Gas detection theory model has been researched for many years, which is mainly proposed through a model of cluster semiconductor sphere with the current between cluster calculated through thermal emission and carrier tunneling. The surface electron density represents the chemical status of the gas adsorbed and the depletion layer width is constructed based on the calculation of abrupt junction model with the consideration of surface status of consistent semiconductor energy band. The sensitivity is calculated through logarithm of surface density and conductivity. Gas detection mainly uses the change of electrical property at different locations due to chemical adsorption effect, and the most commonly used model is to use the strength of the adsorbed particles for qualitative analysis, which involves the conversion relation among gas and solid by charges of conduction electron due to adsorption effect (donor or acceptor).

In the commonly used metallic oxide sensor, factors such as the existence of oxygen or the reduction of background gas in the environment will all have very obvious effect on the change of electronic conductivity. It can not be purely explained by the change of the conduction electron concentration, the change of energy gap of potential energy formed by the adsorbed acceptor or donor at the interface should be considered at the same time, which in turn controls the electron flow on both sides of the junction. When N-type semiconductor oxide is exposed to environment that contains reduction gas, the adsorbed oxygen will gradually be consumed due to the reaction with reduction gas. The reduction of oxygen ion on the surface of semiconductor oxide will let the electrons trapped by oxygen go back to the crystal grain. This process will lead to the reduction of energy gap, that is, a reduction in the resistance.

When the gas to be tested is in contact with semiconductor, energy level will be changed. Electron will flow from high Fermi energy level area (the area in the neighborhood of semiconductor surface) to low Fermi energy level area (surface status). The separation of electronic charge will lead to the formation of double layer voltage, which in turn increases the surface energy. When the double layer voltage is large enough to let the Fermi energy level of the entire system become a constant, an equilibrium state is reached. The band movement close to the surface is called “band bending”. This phenomenon is used to represent the adsorption of gas on the crystal surface which in turn cause the change of the surface status; moreover, this phenomenon will cause the change of characteristic of carbon nanotube, for example, electrical inductance, electrical conductivity (or electrical resistance), dielectric constant and mass. Therefore, the measurement of the electrical property after the reaction of carbon nanotube with specific gas can be used as the method to detect the gas.

### Carbon Nanotube Temperature Detecting Principle

Wong et al. had used batch fabrication for the carbon-nanotube-based thermal sensors [IEEE trans. Automation Science and Engineering, Vol. 3, 3 Jul. 2006, 218-227]. They used Dielectrophoresis (DEP) force manipulation technology to place carbon nanotube across two electrodes with better deployment orientation and contact so as to form a loop and to conduct the electric current, and carbon nanotube here is used as resistance sensor unit to detect the temperature change; furthermore, it is found in the experiment that the temperature coefficient of resistivity (TCR) shows a negative slope, that is, the resistance of carbon nanotube will fall along with the rise in temperature; from the voltage and current measurement, it shows that the power consumption of carbon nanotube is calculated to be about in the range of μW, and the room temperature resistance distribution scope is from several KΩs to several hundreds KΩs. Li et al. had used defined microstructure and the manipulation technology of Dielectrophoresis (DEP) force and carbon nanotube to form a resistor unit, the experimental result shows that the self-heat current needed by carbon nanotube is much smaller than that needed by traditional polysilicon material made by MEMS technology. In addition to that, this device, under constant current mode, has faster frequency response (>100 KHz); meanwhile, it can be used as hot-film anemometry and the power needed by this flow sensor is about 15 μW.

### Manufacturing Technology of Carbon Nanotube Sensor

The synthesis and application of carbon nanotube can be generally divided into the following preparation ways: (1) Arc-discharge method; (2) Laser ablation method; (3) Chemical vapor deposition method. Most of the articles in the literature use chemical vapor deposition method to prepare carbon nanotube sensor device on the substrate with a needed deposition temperature of about 600 degree C. However, at room temperature, the present invention can use DEP force to place carbon nanotube across the electrodes and measure the resistance or dielectric constant of carbon nanotube or prepare a transistor and measure the electrical property; if making surface modification first on carbon nanotube and then placing it across electrodes using DEP force, the present invention can then measure specific gas.

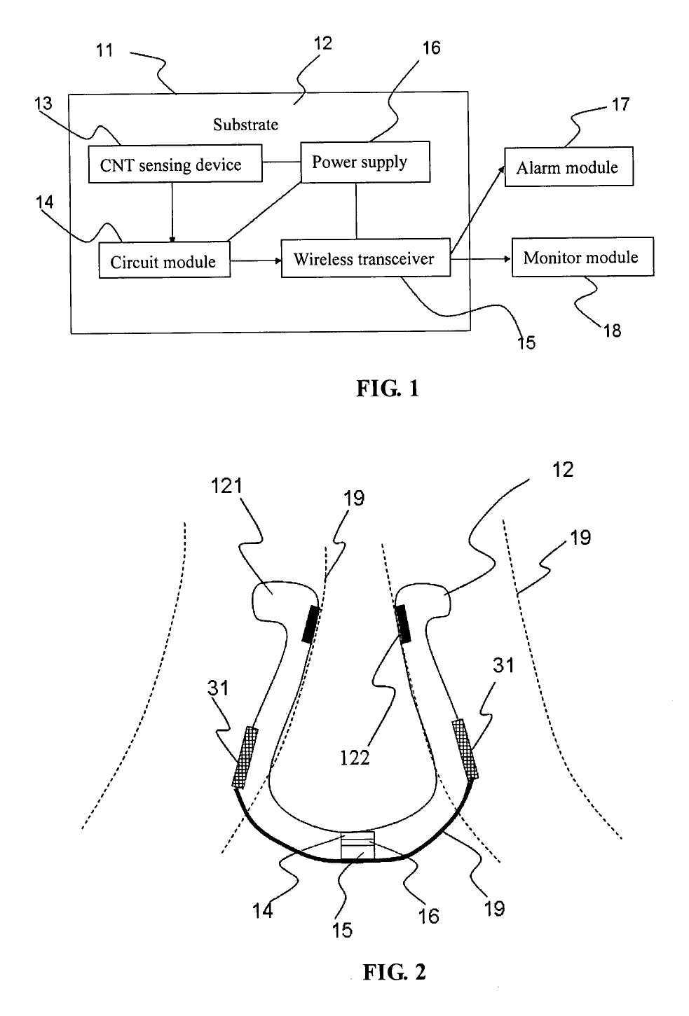

Please refer to FIG. 1, it can be seen from the figure that a device 11 that can detect the gas and aerosol inhaled and exhaled by human body is proposed in the present invention, which is mainly a substrate 12 that can be clamped on the columella between the nostrils is used to carry carbon nanotube sensor device 13, electronic circuit module 14, wireless transmitting/receiving module 15, power supply 16, alarm device 17 and monitoring device 18.

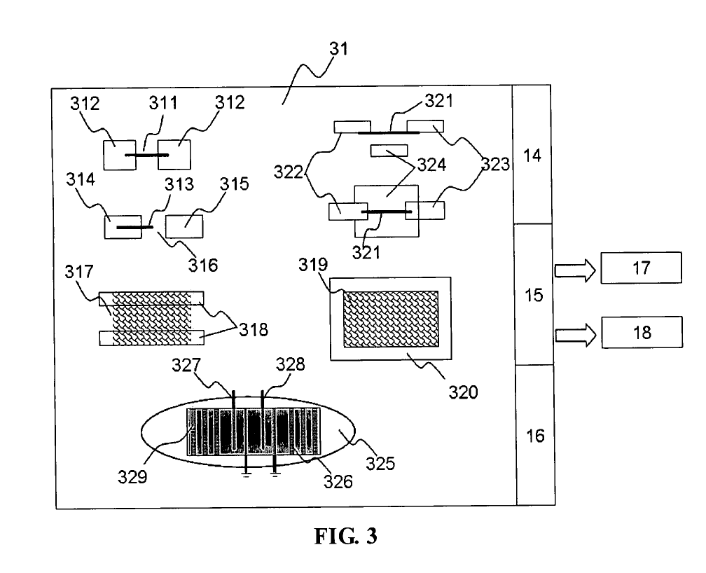

Substrate 12 is bio-compatible polymer material, for example, PHA polymer, Lexan HPX8R, Lexan HPX4, PHBHHx, etc., which possesses clamping structure 121 or adhesion structure 122 so that the device is a structure can be hung on the nose wall 19 of human body, which is as shown in FIG. 2. Meanwhile, we can also make the structure as a nose ring (not shown in FIG. 2) so that it possesses a decorative function at the same time.

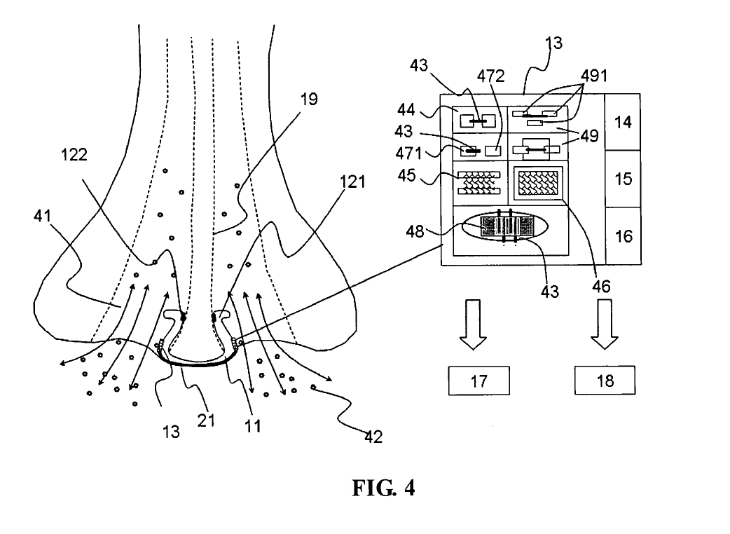

FIG. 3 shows all methods using carbon nanotube as sensor device, Among them, carbon nanotube sensor device 31 is at least one carbon nanotube 311 placed across two electrode structures 312, and the resistance change of carbon nanotube is measured here.

Another carbon nanotube sensor device 31 is at least a carbon nanotube 313 fixed to an electrode 314 and heads toward another electrode 315 and has a spacing of 316 maintained with the other electrode; furthermore, when we apply voltage across both sides, we can measure the breakdown voltage and breakdown current of carbon nanotube.

There is yet another carbon nanotube sensor device 31 which is at least a carbon nanotube 317 installed between capacitor structure 318, then a bias is applied between the capacitor structure and the change in capacitance value and dielectric constant is measured.

There is further another carbon nanotube sensor device 31 which is a network carbon nanotube 319 (CNT network) installed on a dielectric thin film of a capacitor structure to be used as upper electrode 320, and the lower electrode is metal installed below the dielectric thin film to measure the capacitance change.

More another carbon nanotube sensor device 31 is carbon nanotube 321 placed respectively across source electrode 322 and drain electrode 323, and gate electrode 324 in the neighborhood of carbon nanotube 321 forms together with the above mentioned structure a field effect transistor structure that can be used to control the property of carbon nanotube 321 and the transistor characteristics of carbon nanotube can then be measured.

There is furthermore a carbon nanotube sensor device 31 which is at least a carbon nanotube 325 deposited on acoustic sensor 326 so as to measure the resonance frequency change of the acoustic sensor. Moreover, the carbon nanotube 325 can be coated on film body acoustic resonator (FBAR) to measure its resonance frequency change.

To make a conclusion, we know that the above carbon nanotube sensor device 31 is a sensor device that can be used to detect the gas inhaled and exhaled by human body (for example, detecting the gas species, concentration and its rate of change and temperature and humidity) and then send the gas signal into the electronic circuit module 14 for conversion;

The electronic module 14 is adjacent to carbon nanotube sensor device 31, wireless transmitting/receiving module 15 and power supply 16 and used as data processing, conversion and exchange center; the data processing includes magnification of signal, signal filtering, analog/digital signal conversion, signal coding or signal decoding.

The wireless transmitting/receiving module 15 receives the detected gas signal converted from electronic module 14, and uses wireless way, through antenna 21, to send the detected signal of gas and aerosol inhaled and exhaled from human body to remote alarm device 17 or monitoring device 18; furthermore, the human skin can be used as media for transmitting and receiving the signal to send the signal to the warming device or monitoring device (not shown in the figure) worn or attached on other parts of human body, for example, waist and hand.

The warning device 17 is used to receive the detected signal of gas and aerosol inhaled and exhaled from human body sent out from wireless transmitting/receiving module 15. Moreover, warning status can be set up, and warning messages can be sent out in the warning status, for example, by one of the following ways such as: vibration, sound, bright light or a display through a screen, etc.

The monitoring device 18 is used to receive detected signal of the gas and aerosol inhaled and exhaled by human body and sent out from wireless transmitting/receiving module 15, the signal is then monitored and recorded.

Power supply 16 is a battery or wireless power supply module or a power supply module sent through the body surface.

### Manufacturing Technology Integration Between Carbon Nanotube and CMOS Chip

In the present invention, one more method is proposed to deposit in low temperature the carbon nanotube effectively and in large scale and adhere and fix it on the exposed metal of a passivation opening that is previously designed on a CMOS. In order to fix carbon nanotube on the metallic layer, first, take tiny amount of the previously acquired and sorted single wall or multi-wall carbon nanotubes and immerse them in DI (de-ionized) water solution that contains 1-wt % Sodium Dodecylsulfate (SDS) so that the wall of carbon nanotube will be covered by SDS molecules; moreover, carbon nanotube concentration should be diluted to a status depending on application needs, and 0.35-wt % of Ethylene Diamine Tetra Acetic Acid (EDTA) and 4-vol % TRIS-HCl buffer should be added so as to compound the residual transition metal ion and to maintain a stable PH value. First, ultrasonic vibration will be used to vibrate and separate bundled carbon nanotubes, then a centrifugal device is used to let bundled carbon nanotubes that is coated with SDS molecules on the outer wall and impurity precipitate to the bottom; then the low mass single carbon nanotubes with outer wall coated with SDS molecule will be centrifuged to the upper level of the container; then extract carefully the 30%˜80% solution on the upper level of the solution, these carbon nanotubes can then be used for manipulation and fixing. By referring to the literature [Zhi-Bin Zhang et al. “Alternating current dielectrophoresis of carbon nanotubes”, J. Appl. Phys., Vol. 98, 056103, 2005], we can be sure that after carbon nanotube solution is treated by such method, not only the subsequent manipulation of carbon nanotube by Dielectrophoresis (DEP) force is easier. In addition, since semiconducting carbon nanotubes have the different DEP property from metallic CNTs, they are more favorable to be applied in the application of fixing carbon nanotube, that is, manipulation frequency and electrode design as well as channel design can be used to effectively separate the metallic and semiconducting carbon nanotubes.

Drop solution containing carbon nanotubes on the exposed metallic pad above CMOS structure and apply DEP force to manipulate carbon nanotube. Through the adjustment of AC frequency, AC peak-to-peak voltage and DC voltage, we can adjust and manipulate the DEP force of carbon nanotube; meanwhile, at the time of the application of DEP force, we can add impedance meter through a model of lock-in amplifier that, at the same time while the DEP signal is applied, impedance measurement can be performed as well; by doing so, the impedance value can be measured at the same time so as to detect the quantity of carbon nanotube attached on the electrode; In addition, through the use of positive DEP and negative DEP force, the extra or not originally targeted number of carbon nanotube on the electrode are excluded by negative DEP force through the use of the adjustment of AC frequency, AC voltage (Peak-to-Peak voltage), DC voltage, etc.; then perform once again the signal and apply signal of positive DEP force range, until the needed carbon nanotube number is reached, then keep the DEP force until the evaporation of the dielectric solution, and finally, blow in N₂ gas to blow dry the water beads remained on the surface. Therefore, through the use of this method, carbon nanotube can be fixed on the CMOS chip through the use of low temperature post-process, the damage of the CMOS won't be caused due to the high temperature problem as mentioned above; moreover, the number of carbon nanotube associated on the electrode can be effectively controlled. By using lift-off process, a comb-shape metal layer of Cr/Au as electrodes can further be deposited on the area of CNTs to make the CNTs firmly be fastened under the electrodes. Consequently a system type chip processor unit, or called as System-on-Chip (SOC) with carbon nanotubes associated on CMOS structure is then achieved. Here please also note that in the present inventions if the sensor device based on carbon nanotubes after manufacturing has a deviation of electrical characteristics from the specification, we still can employ laser trimming technique to cut out part of the CNTs to adjust the sensed signal to meet the required specification, which is similar to the counterpart in analog integrated circuits industry.

In the followings, the attached drawings and examples will be referred to for the descriptions of the technological means and functions used by the present invention to achieve its goal; the examples as listed in the following figures are only aids for the descriptions so as to facilitate the understanding, but the technological means of the present invention should not be limited by the figures listed.

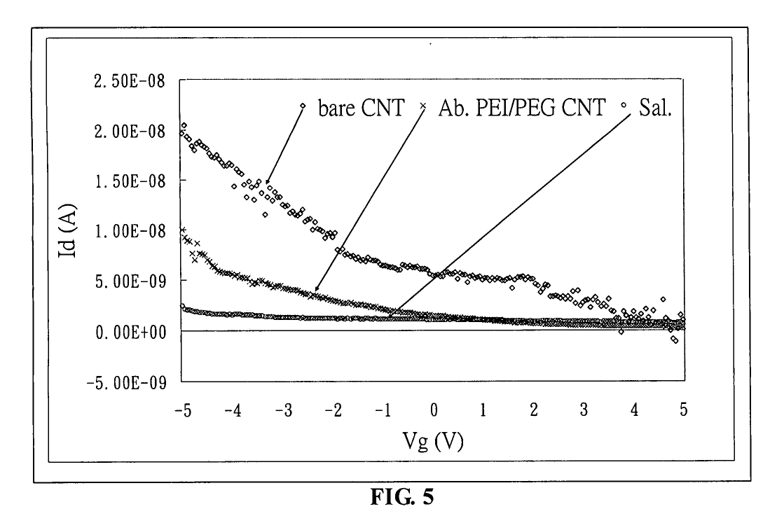

Please refer to FIG. 4, which is an illustration of the device when it is worn on the nose of a person; it can be seen from the figure that the device and system 11 which can detect the gas and aerosol inhaled and exhaled from human body is a structure comprising of a substrate of clamping structure 121 or adhesion structure 122 and is clamped on the columella between the nostrils 19; moreover, its carbon nanotube sensor device 13 is aligned to the breathing gas channel 41 so that carbon nanotube sensor device 13 can get contacted with the gas and aerosol inhaled and exhaled through the nose, then the specific gas molecule and aerosol 42 inhaled and exhaled will get in contact with the surface of carbon nanotube 43 of carbon nanotube sensor device 13 and lead to the change of resistance, capacitance, mass, breakdown voltage and current of carbon nanotube. For example, through the use of the resistance measurement structure 44, we can measure the resistance change of carbon nanotube; through the use of capacitance structure 45, we can measure the dielectric constant change and the above mentioned methods can be used as the measurement of gas concentration and humidity; network carbon nanotube is used as capacitor structure of upper electrode 46, that is, carbon nanotube thin film is coated on the dielectric thin film of a capacitance structure as the upper electrode, and the lower electrode is metal which is installed below the dielectric thin film; then a capacitance change due to the reaction between carbon nanotubes and gas can be used as highly sensitive gas concentration measurement; carbon nanotubes 43 are connected to an electrode 471, and bias voltage is applied at another electrode 472, then the breakdown voltage and current is measured; carbon nanotubes 43 can be coated on surface acoustic waves sensor 48 and the mass or resonance frequency change of acoustic sensor can be measured; combine carbon nanotubes 43 with three electrodes 491 to form a carbon nanotube transistor 49, then, through the measurement of the characteristic change of carbon nanotube transistor, for example, the relationship between gate voltage V_{g} and the drain and source electrode current I_{sd}, we can detect the species and concentration of the gas.

Connect the above mentioned carbon nanotube sensor device 13 with electronic circuit module 14, wherein the circuit has a structure for amplifying circuit signal, filtering out noise and measuring signals (for example, resistance, dielectric constant, capacitance, inductance, resonance frequency, breakdown voltage and transistor characteristics); then through an analog/digital conversion, an wireless transmitting/receiving module 15 then transmits the signal to warning device 17 or monitoring device 18, wherein the wireless transmitting/receiving module 15 receives the gas and aerosol detected signal converted from electronic circuit module 14, and through wireless transmitting method through antenna 21, the detected result of gas and aerosol inhaled and exhaled by human body can be transmitted to warning device 17 or monitoring device 18 through wireless method; when the species of the specific gas and aerosol or the gas temperature exceed the warning range, the monitoring device 18 will monitor and record the gas and aerosol signal inhaled and exhaled by human body, then the warning device 17 will issue warning message, for example, vibration, sound, bright light or a display through a screen, etc. to inform the user or the nursing personnel or convert the carrier signal into digital data and have it displayed in a monitor screen so as to achieve the purpose of monitoring and to inform the user to move away immediately the environment where the gas and aerosol exist and to avoid the possible hurt caused by the harmful gas and aerosol.

In order to let the species of gas detected be more diversified, in addition to using highly specific surface modification method, the sensor can also be an array type sensor, that is, the above mentioned sensor devices can be assembled in multiple ways; in other words, different electronic circuit designs and carbon nanotube devices can be constructed on the same chip, or multiple same carbon nanotube devices can be given with different surface-modified recipes so that it can have different level of reaction with different gases; furthermore, through a classifying algorithm, for example, neural network or principal component analysis (PCA), etc., pattern recognition can then be performed and all kinds of different gases can then be distinguished effectively. Generally speaking, the present invention uses the modified materials that are commonly used for traditional gas sensor, for example, metals (Pd or Au, etc.), polymer, metal oxide, hydrogen-ion or OH-ion-containing material, to perform modification on carbon nanotubes so that it can achieve specific judgment on the biomarker gases exhaled from human body through PCA or neural network algorithm.

In the present invention, electrodes can also be made on standard substrate with electrode patterns other than a CMOS chip. Through the use of masking method, carbon nanotube solution is sprayed or spotting among the electrodes, then the solvent is waited for its natural evaporation to form nano thin film, and finally, all the electrical characteristics of the carbon nanotube thin film are measured and the results are used as comparison reference for bio detection. Since carbodiimidazole-activated Tween 20 (CDI-Tween) is covered on carbon nanotube and its hydrophobic characteristics are used to modify the surface of carbon nanotube; then antibody or aptamer or carbohydrate are going to be combined with CDI-Tween-treated single wall carbon nanotube through covalent bonding. When antibody or aptamer or carbohydrate are successfully adhered to carbon nanotube to modify the carbon nanotube, then the effect and change of antibody or aptamer or carbohydrate on the electrical properties of single wall carbon nanotube transistor (SWNT-FET) will be measured; it is believed that through the use of the special characteristics of antibody or aptamer or carbohydrate, the sensitivity and property of carbon nanotube transistor can be enhanced; furthermore, the antibody or aptamer or carbohydrate are used as carrier to selectively detect the acceptor, that is, carbon nanotube transistor bio sensor device are successfully constructed with antibody or aptamer or carbohydrate as identifying component.

For the detection device for detecting gas and aerosol inhaled and exhaled by human body as proposed in the present invention has the following advantages as compared to other prior art methods:

1. The device and system of the current invention for detecting the gas and aerosol inhaled and exhaled by human body is attached or clamped on the nose wall through clamping structure and can be used to monitor the gas and aerosol inhaled and exhaled by the user for a long time; since it has a small volume and is of light weight, it won't be any load to the user.

2. The current invention is installed at the nose end with the gas directly coming from the inhalation or exhalation of the user, which is quite different as compared to other handheld or fixed or wearing type detection methods; therefore, extra gas pumping device is not needed and the harmful gas or bioaerosol in the environment can be effectively grasped.

3. Since the present invention is installed at the nose end, another advantage of it is that whether bioaerosol that can spread through breathing exist inside of the human body can be known, for example, the flu virus or tuberculosis bacteria, etc., and of course, the specific odor might possibly represent the symptom of certain disease.

4. The present invention detects the gas through the measurement of one of the following properties on the carbon nanotube sensor device, for example, resistance, dielectric constant, resonance frequency, transistor characteristic and breakdown voltage, etc., the accuracy of gas detection can be greatly enhanced.

5. In the present invention, DEP force is used to assemble semiconductor carbon nanotube onto specific electrode; since the carbon nanotube is prepared separately with the electronic circuit, hence, before the assembly of carbon nanotube, carbon nanotube can be separated in terms of metallic type and semiconductor type or surface modification (doping) can be done to enhance the sensitivity and specificity of gas detection.

6. In the device and system of the present invention for detecting the gas and aerosol inhaled and exhaled by human body, the electronic circuit module can be manufactured through the use of standard CMOS process and the batch manufacturing of this device and system is thus feasible.

7. In the device and system of the current invention for detecting the gas and aerosol inhaled and exhaled by human body, since it can be used together with mobile phone or wireless communication, the monitoring distance can then be enhanced.

### EXAMPLE 1

### Detection of the Change of NO Exhaled by the Human Body

The management of inflammation of respiratory tract is dependent on appropriate monitoring and curing so as to obtain a long term effect. However, the current method has its limit; therefore, it is very difficult to achieve such goal. Although nitric oxide (NO) has been identified early 200 years ago, yet its physiological importance is really recognized in the beginning of 1980s.

Many researches have identified NO as one of the major message molecules inside the body system, in addition, many researches also find that the change of NO exhaled is highly related to other markers of the inflammation of respiratory tract. Since the technology of the measurement of NO exhaled is non-invasive, reproducible, sensitive, and easy to implement; therefore, the monitoring of the exhaled NO change can be used to manage asthma and other lung disease. [Choi J et al., Markers of lung disease in exhaled breath: nitric oxide, Biological Research for Nursing, 2006 April, 7(4):241-55.]

Carbon nanotube is first placed in the reflux of H₂O₂ and a mixing solution of sulfuric acid and nitric acid (3:1) so as to remove carbon nano particle and to generate functional group on the carbon nanotube to be used as place for the covering of SnO₂; next, place this acid-treated carbon nanotube in 80 mL and 0.1 mol/L tin(II)chloride solution and add 1.4 mL of HCl, then use ultrasonic vibration to agitate for 30 minutes, then filter the product and use distilled water to clean it. By doing so, the nanoparticle of SnO₂ will be coated uniformly on carbon nanotube with dimension of about 2-6 nm.

Then connect the surface-modified carbon nanotube through DEP force to between two electrodes to complete impedance type or transistor type device or the device of the detection method as proposed by the present invention.

The operation principle is: When sensor is in the air, oxygen molecule will be absorbed to SnO₂ nanoparticle and extract electrons from the SnO₂ nanoparticle to become oxygen ion and SnO₂ will in turn carry positive charge and barriers will be formed among SnO₂ nanoparticles. Since the SnO₂ nanoparticle is very small, there are thus a lot of interstitials to absorb the gas and react with it. When the sensor is placed in NOx gas, easily oxidized gas molecule will be further adsorbed onto SnO₂ nanoparticle and extract electrons to form higher barriers; therefore, for resistance type sensor, the resistance is going to rise a lot; however, when the sensor is placed back into the air again, NOx molecule will be released again from SnO₂ nanoparticle and the electrons will be released back too; therefore, the resistance of the sensor will go back to the original value in the air.

### EXAMPLE 2

### Detection of Biological Aerosol

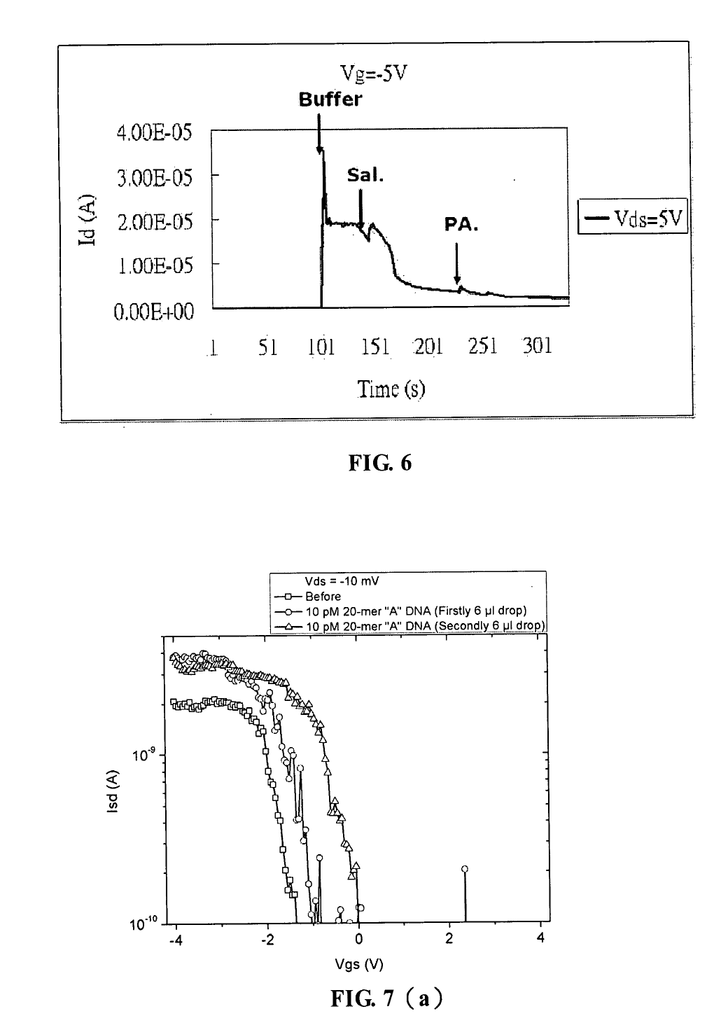

Using cross-linking agent to modify antibody onto the carbon nanotube surface, then the carbon nanotube transistor is used to detect biological particle so as to understand the electrical property change when biological particle is attached to the surface of carbon nanotube. The characteristic diagram of I_{sd}−V_{gs} is shown in FIG. 5, in the figure, bare CNT represents carbon nanotube transistor that is not modified, ab PEI/PEG CNT represents the use of PEI/PEG to modify the antibody onto the surface of the carbon nanotube transistor; from FIG. 5, it can be seen that after antibody modification, the transistor I-V characteristic curve has a trend to move horizontally to the left, and the Vg (Threshold Gate voltage) moves from original 5V to the left horizontally to about 1V, the reason is because electrons are transferred to the carbon nanotube through protein, which in turn leads to the horizontal shift of I-V characteristic curve.

In FIG. 5, Sal. represents the transistor I-V characteristic curve after Salmonella is combined with carbon nanotube, as compared to that before a combination with Salmonella, it can be seen that there is a dramatic drop in the current, and the threshold gate voltage Vg is maintained at about 1V. The change is because when antigen is combined with the antibody on the surface of carbon nanotube, the surface of the carbon nanotube will get distorted and the surface charge migration of the carbon nanotube will get reduced and I-V characteristic curve will get reduced too.

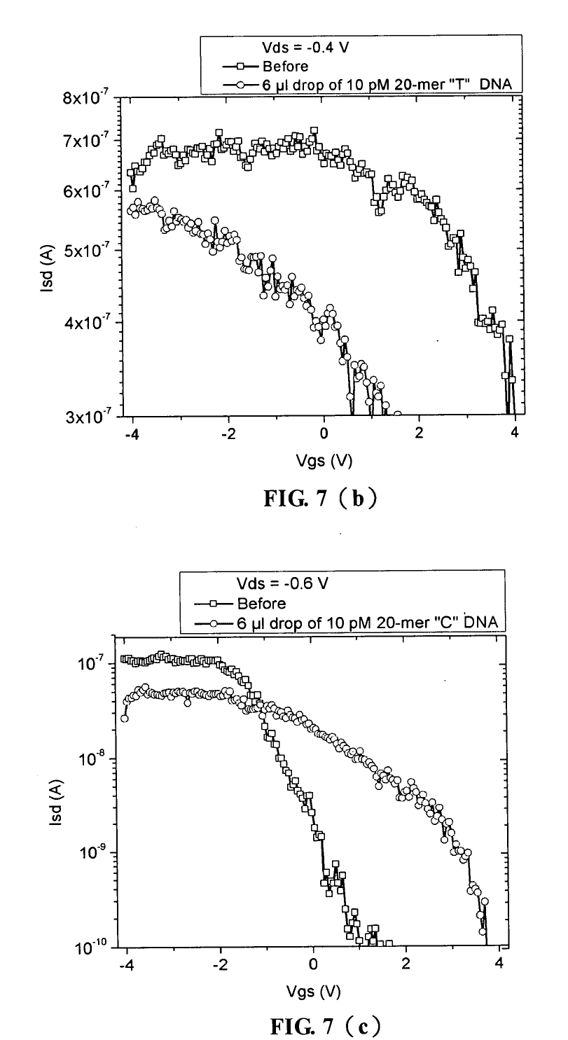

When the transistor source and drain electrodes are applied with a bias voltage Vds of 5V and a gate voltage of 5V is fixed, the current shows a change as in FIG. 6, which is a real time current signal measurement of a transistor. In the beginning, when the carbon nanotube transistor is applied with Vds bias of 5V and −5V of gate bias voltage, transistor will maintain at a current of 8×10⁻⁸ A, and when PBS buffer solution is dropped between the two electrodes, a water bead will be formed to enclose the carbon nanotube because of surface tension, at the moment while the liquid is dropped, there will be a surge current, then the current will go back and maintain stably at 2×10⁻⁶ A; later on, mix PBS mixed solution with Salmonella on the liquid drop, we will find an obvious drop on the current, when it drops to about 1.4×10⁻⁶ A, it will remain stably; later on, add other types of cells (Pseudomonas aeruginosa) into the buffer solution, the current won't change. Therefore, through such an electrical signal experiment, it can be found that when Salmonella combines with antibody, the electrical conductivity of carbon nanotube will get dropped obviously.

The above mentioned Salmonella can be changed to Mycobacterium tuberculosis, or flu virus, and the related antibody should also be changed, then the flu virus or aerobic bacteria which spreads in the air can be detected.

### EXAMPLE 3

### Detection on the Change of Acetone by DNA Modified CNTFETs

The major symptom of diabetes is high blood glucose concentration. Therefore, the patient can not take full use of glucose, at the same time, the decomposition of fat will be accelerated and fatty acid will be generated, which in turn is converted into ketone bodies. If the ketone bodies generated are limited, it can be used by the tissue, for example, the muscle tissue; however, if the ketone bodies generated is too much, it can not be fully used by the tissue, it will then be released as ketonuria; therefore, the exhaled gas will smell like rotten apple with acetone-like odor. The present invention can be used to do early stage detection of diabetes in human body in real time way and in the long term; or to do monitoring to see if it get worse after an early stage detection; or to check if the cure is effective; it can be used as a reminder for taking medicine.

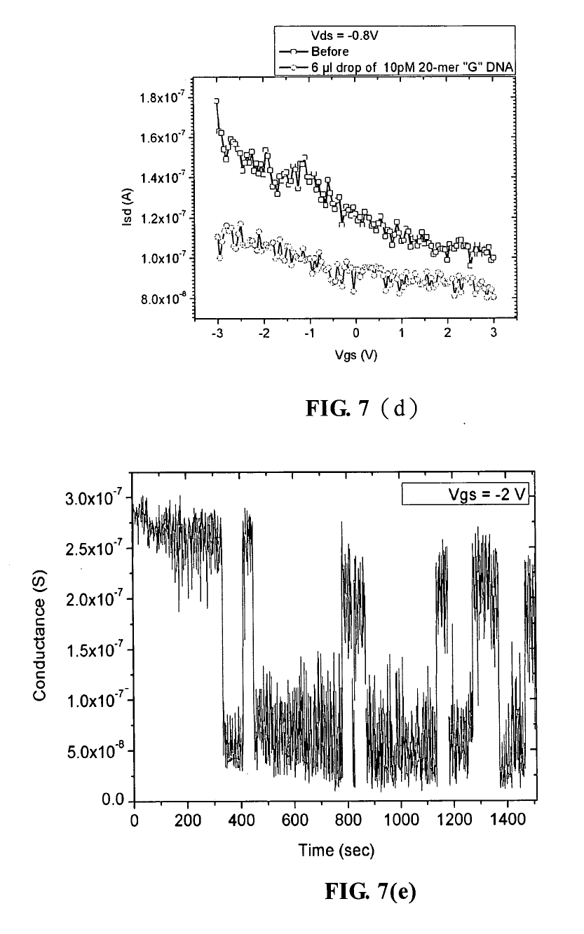

FIG. 7 shows that through carbon nanotube field effect transistor and through the combination of single-strand DNA and carbon nanotube, we can measure acetone effectively.

As previously described fabrication process of CNTFETs, during the purification and separation of carbon nanotube, carbon nanotube will be immersed in SDS solution so as to coat SDS on the surface of carbon nanotube; however, when carbon nanotube coated with SDS is adhered to the electrode, the contact between carbon nanotube and the electrode metal is for sure going to be affected by SDS; therefore, the removal of SDS coated on carbon nanotube is a key to optimize device characteristics.

After the completion of the adherence of carbon nanotube and the measurement of I_{sd}−V_{gs} characteristics as well as the removal of SDS, CNTFETs with good characteristics can then be used in the sensor application. In the following, CNTFET with good characteristics is to be used for the detection of DNA and acetone and the electrical property change is going to be measured. As shown in FIG. 7, CNTFET is used as biomedical sensor (ssDNA) and gas sensor (acetone). FIG. 7 (a) is the titration of “A” basic ssDNA, “ON” current will rise and the I_{sd}−V_{gs} curve will shift toward “positive” direction; FIG. 7 (b) is the titration of “T” basic ssDNA, “ON” current will drop and I_{sd}−V_{gs} curve will shift toward “negative” direction; FIG. 7 (c) is the titration of “C” basic ssDNA, “ON” current will drop and I_{sd}−V_{gs} curve will shift towards “positive” direction; FIG. 7 (d) is the titration of “G” basic ssDNA, “ON” current will drop; FIG. 7 (e) is the real time measurement of acetone by CNTFET sensor which is surface-modified with DNA; it can be seen that the current invention has very sensitive reaction and very high signal to noise ratio.

As shown in the literature [Lu, Yijiang; Partridge, Christina; Meyyappan, M.; Li, Jing, “A carbon nanotube sensor array for sensitive gas discrimination using principal component analysis” Journal of Electroanalytical Chemistry Vol: 593, Issue: 1-2, Aug. 1, 2006, pp. 105-110], the present invention uses modified materials which are commonly used in traditional gas sensor, for example, metals (Pd or Au, etc.), polymer, metal oxide or substances containing hydrogen ion or OH ion, to perform modification on the carbon nanotube; meanwhile, an array is formed to perform and achieve specific judgment on all kinds of biomarker gases exhaled by human body through PCA or neural network algorithm; here we take example on the acetone generated by diabetes patient, the present invention can detect in real time and continuously the acetone exhaled by diabetes patient in very early stage and in very low concentration so that the patient can be cared in early stage. Take an example on the volatile organic compound (VOC) such as acetone, the present invention can also employ traditional polymer material, for example, chlorosulfonated polyethylene and hydroxypropyl cellulose polystyrene, polyvinylalcohol, etc. (which is commonly used in the polymer-based organic gas sensor available in the market) to achieve its purpose.

### EXAMPLE 4

### Detection of Flu Virus

Since carbon nanotube is ideal material for ultra small sensor and its ultra large surface area has very high sensitivity on the transfer of electronic charge. High quality single-wall carbon nanotube transistor (SWNT-FET) is used to be combined with flu aptamer, and array method is used for the deployment to increase the contact opportunity between the droplet containing flu virus vaccine and the flu aptamer on the surface of the carbon nanotube so as to enhance the detection sensitivity.

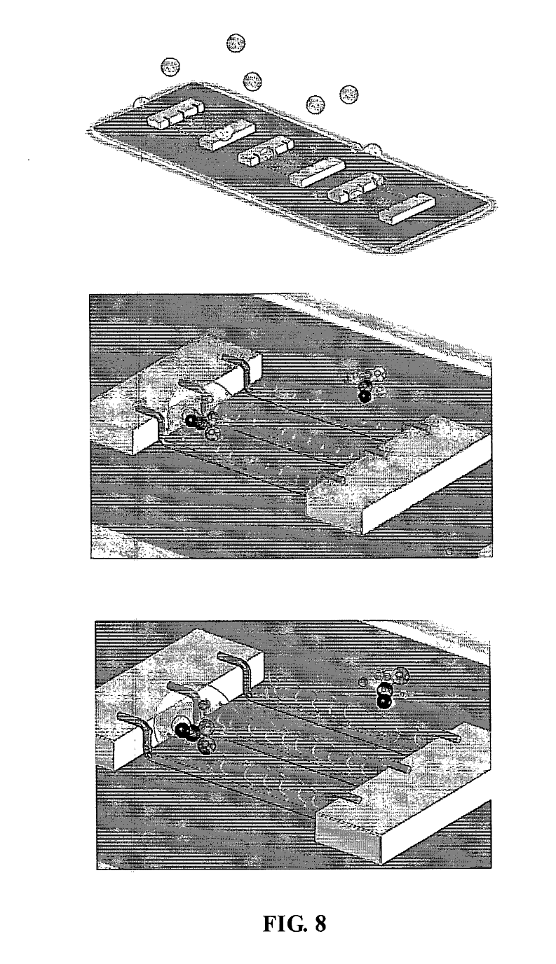

Immerse the flu virus vaccine in dielectric solution through KCL solvent or mannitol with a main purpose to change the dielectrophoretic property to facilitate the manipulation. First, electrodes are prepared on the glass substrate, and the above mentioned DEP force is used to manipulate carbon nanotube on the Source and Drain of the transistor. Take several drops of KCL or mannitol solution containing flu virus vaccine with micro titrator and drop it on the glass substrate that is prepared with electrode and carbon nanotube, then use a lead to connect it to the display. Use optical microscope to observe the current curve of the solution before the adding of flu virus vaccine solution as the reference group. Then add the flu virus vaccine containing solution to the carbon nanotube to let flu virus vaccine adhere to carbon nanotube and observe the current change at the externally added display, the illustration is as shown in FIG. 8.

FIG. 8 shows that droplet containing flu vaccine could get close to CNTFETs chip due to the suction action (for example, the suction action of human nose); when it gets in contact with flu antibody or flu aptamer of multiple single-wall carbon nanotubes (only single nanotube is shown in the figure.), since a droplet can contain several flu viruses and the droplet size is about 1-5 um which can enclose the reaction range of flu virus and flu antibody or flu aptamer; that is, the binding environment and condition of virus and antibody or aptamer is in the solution, this matches the condition of water solution for the original manufacturing and artificial synthesis of antibody or aptamer, hence, it has very high specificity and sensitivity.

For the experiment on other viruses, it is similar to the above mentioned steps and the only difference is the antibody on the carbon nanotube.

The above detailed descriptions are only some of the possible embodiments of the current invention and the embodiments are not to be used to limit the scope of the current invention, any equivalent embodiment or change that does not depart from the technological spirit of the current invention should all fall within the scope of what is claimed.

All publications, patent and patent applications cited herein are incorporated herein in their entirety by reference.

What is claimed is:

1. A device for detecting gas and aerosol inhaled and exhaled by human body, comprising of:
(1) a substrate, which is used to carry gas and aerosol sensor unit, electronic circuit module, wireless transmitting/receiving module, power supply and a structure for hanging around the nose wall;
(2) at least one carbon nanotube sensor unit, which is used to react with the gas and aerosol to be detected;
(3) an electronic circuit module, which is installed with electronic circuit layout and is connected to said substrate, said carbon nanotube sensor unit and wireless transmitting/receiving module to perform a special processing on the electrical signal obtained from the measurement of electrical property of said carbon nanotube sensor device;
(4) a wireless transmitting/receiving module, which is used to receive the gas and aerosol signal converted from said electronic circuit module and wireless transmitting method is used to transmit the signal to warning module or monitoring module; and
(5) a power supply, which is electrically connected to said electronic circuit module so as to provide power to said electronic circuit module and said wireless transmitting/receiving module.

2. The device of claim 1 wherein the substrate is installed with at least one of the claming structure or adhesion structure to be used to clamp or adhere the carbon nanotube gas and aerosol sensor unit onto the nose wall.

3. The device of claim 1 wherein the substrate is made up of bio-compatible polymer material, for example, PHA polymer, Lexan HPX8R, Lexan HPX4, PHBHHx, etc.

4. The device of claim 1 wherein the gas and aerosol sensor unit is formed by placing at least one carbon nanotube across two metallic electrodes and it will react with the gas and aerosol exhaled or inhaled by human body to generate electrical property change such as the change of resistance, capacitance and inductance, etc.

5. The device of claim 1 wherein the gas and aerosol sensor unit means at least one carbon nanotube is placed across three metallic electrodes to form a carbon nanotube transistor so as to react with the gas exhaled or inhaled by human body to generate transistor characteristic change.

6. The device of claim 1 wherein the gas and aerosol sensor unit means at least one carbon nanotube that is placed across and coated on a metallic electrode; meanwhile, it will react with the gas exhaled or inhaled by human body to generate resonance frequency change; and the resonance frequency can be measured by surface acoustic wave measurement or/and inductance measurement.

7. The device of claim 6 wherein the carbon nanotube falls within one of the following carbon nanotube categories, that is, a purified single-wall carbon nanotube, a purified multi-wall carbon nanotube, surface-modified single wall carbon nanotube, surface-modified multi-wall carbon nanotube.

8. The device of claim 1 wherein the electronic circuit module further comprising of several integrated circuits (IC) units.

9. The device of claim 8 wherein the integrated circuits (IC) unit is active and/or passive integrated circuits (IC).

10. The device of claim 1 wherein the special processing comprises of at least one of the followings: signal amplification, signal filtering, analog/digital signal conversion, signal coding and signal decoding.

11. The device of claim 1 wherein the wireless transmitting/receiving module uses a wireless transmitting method that is one of the wireless RF technology or the technology with transmission through human skin.

12. The device of claim 1 wherein the wireless transmitting/receiving module comprising of at least an antenna and the antenna is located at the metallic pattern of said substrate and is used to transmit or receive wireless electrical signal.

13. The device of claim 1 wherein it further comprises of a warning module which is used to remind the user, during the breathing action, the type, concentration, temperature and humidity of the gas and aerosol exhaled or inhaled.

14. The device of claim 1 wherein the power supply is battery or wireless power supply module or power supply module that is sent through the body skin.
